(19) United States
(12) Patent Application Publication
(10) Pub. No.: US 2007/0032884 A1
(43) Pub. Date: Feb. 8, 2007

(54) Cable lock device for prosthetic and orthotic devices

(75) Inventor: Bradley Delton Veatch, Westminster, CO (US)

Correspondence Address: SHERIDAN ROSS PC, 1560 BROADWAY, SUITE 1200, DENVER, CO, 80202, US

(73) Assignee: ADA Technologies, Inc., Littleton, CO (US)

(21) Appl. No.: 11/455,064

(22) Filed: Jun. 15, 2006

Related U.S. Application Data
(60) Provisional application No. 60/691,377, filed on Jun. 17, 2005.

(51) Int. Cl. A61F 2/54

(52) U.S. Cl. 623/64

(57) ABSTRACT

A cable lock device includes a body 204, 616 and a frictional shoe 208, 608 operable to engage a Bowden cable 120, wherein, in a first mode, the Bowden cable 120 moves freely in first and second opposing directions 304, 308 and, in a second mode, the shoe inhibits the Bowden cable from moving in the first direction 304 while allowing the Bowden cable to move freely in the second direction 308.

### CROSS REFERENCE TO RELATED APPLICATION

The present application claims the benefits of U.S. Provisional Application Ser. No. 60/691,377, filed Jun. 17, 2005, entitled “Electromechanical Cable Lock for Prosthetic and Orthotic Devices”, which is incorporated herein by this reference.

### FIELD OF THE INVENTION

The invention relates generally to prosthetic and orthotic devices and particularly to cable locks for such devices.

### BACKGROUND OF THE INVENTION

Prosthetic devices, particularly for upper extremity prosthetics, typically include a Bowden cable to control a terminal device to enable the user to grip and release objects. Prosthetic devices are generally of two types, namely voluntary opening and closing devices. In voluntary opening (VO) devices, the terminal device is normally closed. To open the device, the user uses scapular abduction, elbow flexing, or other gross body movements to apply cable tension to the Bowden cable, thereby opening the terminal device. By relaxing his shoulders, the user closes the device. In voluntary closing (VC) devices, the terminal device is normally open. To close the device, the user uses scapular abduction, elbow flexing, or other gross body movements to apply cable tension to the Bowden cable, thereby closing the terminal device. By relaxing his shoulders, the user opens the device.

Users of voluntary opening and voluntary closing terminal devices are plagued by a number of problems. In voluntary closing devices, the gripping digits in the terminal device are splayed open while the unit is at rest, making the unit susceptible to striking nearby objects, and people, as the user moves about. In both voluntary opening and closing devices, users can become fatigued maintaining a selected grasp force over extended periods.

Two methods are currently used to “lock” VC terminal devices closed. In the first method, a bead attached to the cable fits into a small socket cup attached to the prosthesis. The bead keeps the cable from moving axially in any direction to relax grasp or open. In the second method, a pin-and-hole arrangement is used to maintain a closed position. Both of these methods lock the device in only one or at most a few positions (usually closed), restrict movement in both axial directions, and are not useful for effecting or sustaining grasp.

### SUMMARY OF THE INVENTION

These and other needs are addressed by the various embodiments and configurations of the present invention. The present invention is directed generally to a cable locking device and method that is particularly useful for prosthetic and orthotic devices.

In one aspect of the present invention, a method for operating a prosthetic and/or orthotic device is provided that includes the steps:

(a) manipulating a cable lock device to be in a first mode, the first mode allowing a Bowden cable to move freely in first and second opposing directions; and

(b) manipulating the cable lock device to be in a second mode, the second mode inhibiting the Bowden cable from moving in the first direction but allowing the Bowden cable to move freely in the second direction.

The cable lock device can include a platen and a friction shoe positioned on either side of a section of the Bowden cable and an over-the-center spring member (or any other bi-stable mechanism) engaging the shoe. The surface of the shoe engaging the cable is arcuate in shape, and the shoe rotates about a kingpin. The over-the-center spring member biases the shoe against the Bowden cable in the second mode. In the first mode, the shoe is rotated out of contact with the cable.

The shoe is preferably “self-energizing”. In other words, the shoe and cable interaction satisfy the following equation:

Tangent α≦μ, where α is an angle between the kingpin and a point of contact of the shoe with the cable and μ is the coefficient of friction between the shoe and cable.

In a hybrid (or part mechanical/body powered and part electrically powered) embodiment, the cable lock device further includes a lever having an embedded magnetic member. The shoe and lever rotate with respect to one another, and one or more electromagnets displace the lever between first and second positions. When the lever is in the first position, the device is in the first mode, and, when the lever is in the second position, the device is in the second mode.

In a preferred configuration, the cable lock device includes first and second spaced apart electromagnets. The magnetic member in the lever is a permanent magnet, and the shoe and lever rotate about a common axis of rotation. The over-the-center spring member engages both the lever and the shoe. The lever is bi-stable, and the first and second electromagnets are electrically connected in series. When current flows through the electromagnets in one direction, the lever is displaced towards the first electromagnet and, when the current flows through the electromagnets in an opposing direction, the lever is displaced towards the second electromagnet.

The permanent magnet in the lever can be a rare earth magnet. In this configuration, the face of the magnet is covered by a diamagnetic material to provide a space between the magnetic member and a contacting electromagnet. The diamagnetic material can be an elastic, elastomeric, open or closed cell foamed, polymeric, and/or carbon-containing material or composites thereof. The material can provide shock absorption to prevent damage to the magnet from impacts against electromagnets as the lever moves between the first and second modes.

The electromagnetic two-state toggle configuration can provide a compact, energy efficient, and easily controlled single device. The unit can be simple, commonly using only three moving parts (including the over-the-center spring that also moves) and requiring no gears or electric motors. The unit can require electrical energy expenditure only to switch between the first (unlocked) and second (locked) modes or states. This can make the device energy efficient, a desirable aspect for battery operation. Because the device can be simple mechanically, it can also be made robust and lightweight, important considerations for use on a prosthetic or orthotic device that will be worn on the body. It is the mechanism's small size, potential for battery operation, and the fact that it commonly uses no energy unless changing from locked to unlocked or vice-versa that can make it an energy efficient device attractive for prosthetic (or orthotic) applications.

The cable lock device can include safety features to protect the user against a catastrophic, or unexpected, event. In one configuration, the platen is spring-loaded, whereby, when a force exerted by the cable on the platen exceeds a selected level, the platen is displaced, thereby permitting the cable lock device to enter automatically the first mode, from the second mode. In another configuration, the shoe includes first and second bores separated by a projection. The kingpin is in the first bore and separated from the second bore by the projection. When a force exerted by the cable on the shoe exceeds a selected level, the projection fails and the kingpin moves into the second bore, thereby permitting the cable lock device to enter automatically the first mode from the second mode. In yet another configuration, the kingpin includes a stress riser (or a discontinuity or irregularity), whereby the kingpin fails when the force exerted by the cable on the shoe exceeds a selected level, thereby permitting the cable lock device to enter automatically the first mode from the second mode.

These and other advantages will be apparent from the disclosure of the invention(s) contained herein.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

The above-described embodiments and configurations are neither complete nor exhaustive. As will be appreciated, other embodiments of the invention are possible utilizing, alone or in combination, one or more of the features set forth above or described in detail below.

### BRIEF DESCRIPTION OF THE DRAWINGS

FIG. 1 is a side view of a voluntary opening prosthetic device according to an embodiment of the present invention;

FIG. 2 is a disassembled view of a mechanical cable lock according to a first embodiment of the present invention;

FIG. 3 is a plan view of the cable lock of FIG. 2 (with the cover plate removed) engaging, in a locked position, a Bowden cable of the prosthetic device;

FIG. 4 is a plan view of the cable lock of FIG. 2 (with the cover plate removed) engaging, in an unlocked position, a Bowden cable of the prosthetic device;

FIG. 5 is a perspective view of the cable lock of FIG. 2 with the cover plate attached to the face of the lock;

FIG. 6 is a perspective view of an electromechanical, or hybrid, cable lock, in a locked position, according to a second embodiment of the present invention;

FIG. 7 is a perspective view of the hybrid cable lock, in an unlocked position, according to the second embodiment;

FIG. 8 is a plan view of the hybrid cable lock according to the second embodiment;

FIG. 9 is an electrical circuit diagram for the control system of the hybrid system of the second embodiment;

FIG. 10 depicts a toggle arm of the hybrid system according to yet another embodiment;

FIG. 11 is a cross-sectional view through the shoe and platen showing a mechanical cable lock according to another embodiment;

FIG. 12 is a plan view of a shoe according to yet another embodiment;

FIG. 13 is a plan view of a mechanical cable lock according to yet another embodiment; and

FIG. 14 provides a mathematical description of the self-energizing state of the shoe used in various embodiments of the subject invention.

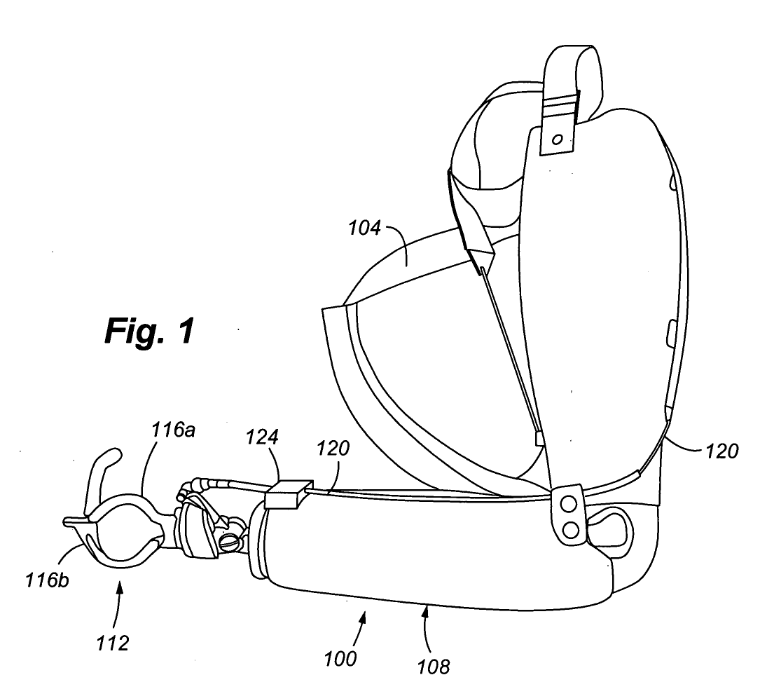

### DETAILED DESCRIPTION

Referring to 1, a prosthetic device according to an upper extremity prosthetic embodiment of the present invention is depicted. The device 100 includes a shoulder harness 104, a forearm assembly 108, and a terminal device 112. The terminal device 112 includes a plurality of digit members 116a and b controlled by a Bowden cable 120 connected, via the harness 104 to the user's shoulder(s). The forearm assembly 108 includes a cable lock device 124 that permits the user to lock the cable in a selected position to prevent movement of the cable and provide a desired positioning of the digit members 116a,b and/or gripping force on an object.

Locking of the cable in the selected position is particularly desirable for voluntary closing (VC) terminal devices. When the cable is locked, the user is able to relax their tension on the control cable without the terminal device releasing its grasp and allowing gripped objects to drop. The grasp can be maintained without cable tension so that users do not get tired trying to sustain high tensions for long periods of time. This is a common complaint that makes VC units less popular among upper-extremity amputees. The voluntary closing terminal device can be locked in a “closed” position with its gripping digits together. This can eliminate the problem of transporting a terminal device with the digits apart, as they look peculiar and tend to strike nearby objects. These benefits can effectively negate the two primary disadvantages of voluntary opening terminal devices, making voluntary closing units equipped with the subject invention more desirable.

As will be appreciated, the cable lock described herein may be used on other devices, such as orthotic devices.

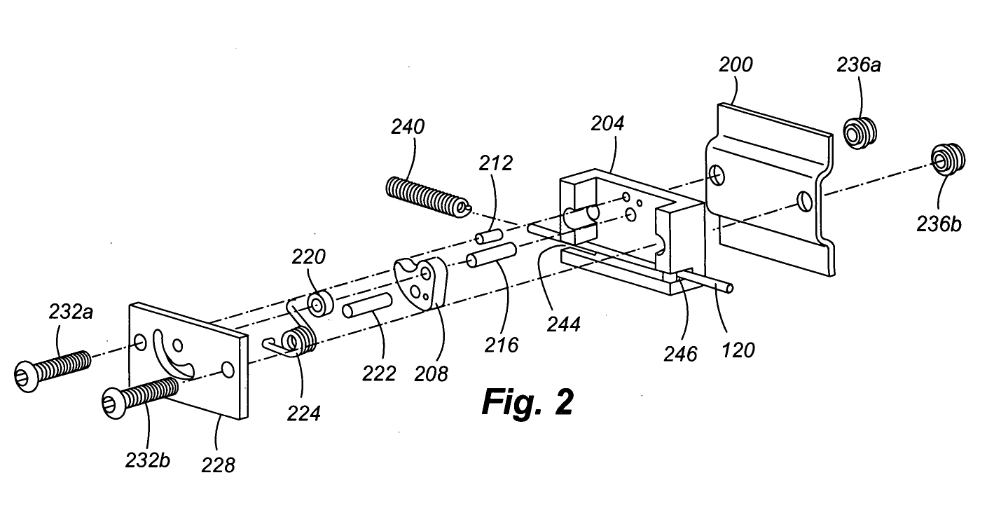

A first embodiment of cable lock device 124 will be discussed with reference to FIGS. 2-5. The cable lock device 124 includes a mounting plate 200, a body 204 engaging the Bowden cable 120, a friction shoe 208 for locking the Bowden cable 120 in a desired position, a movement limiter 212 to limit rotational movement of the shoe, a kingpin 216 about which the shoe rotates, a restraining member 220 to engage the kingpin 216 and hold the shoe in position on the kingpin 216, an actuation lever 222 to permit the user to lock and unlock the shoe against and from, respectively, the cable 120, an over-the-center toggle spring member 224 to bias the shoe against the cable 120, a cover plate 228, and fastening screws 232a,b that engage nuts 236a-b and hold the cover plate 228 on the body 204. The spring member 240 engages the cable inlet cable guide 244 of the body 204. The body 204 further includes a cable outlet guide 246.

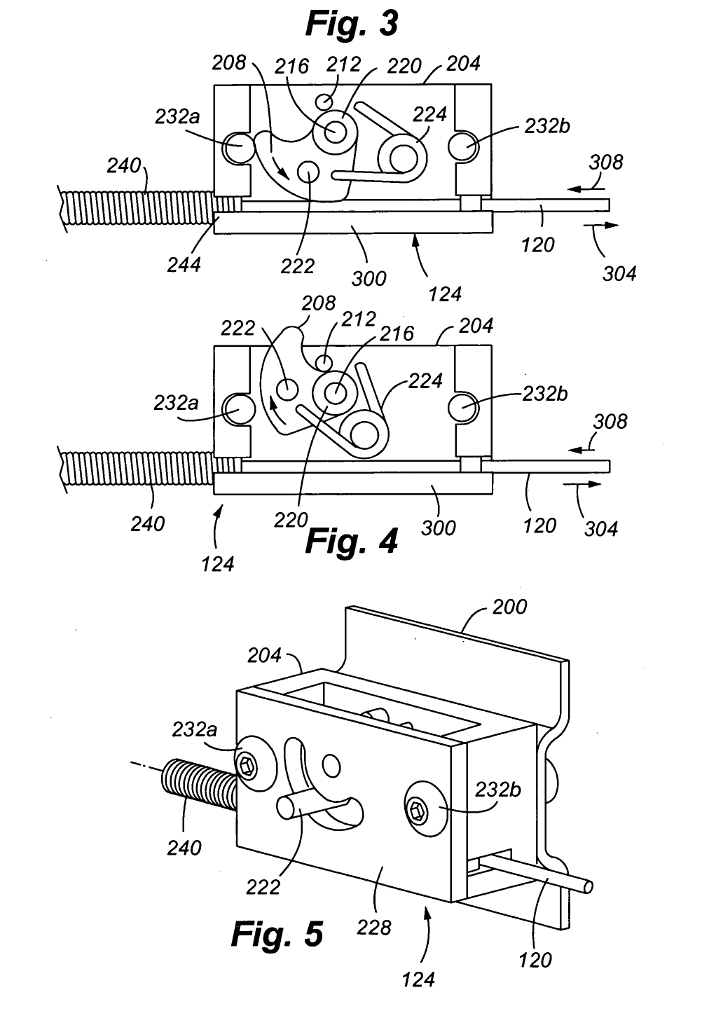

FIG. 3 shows the shoe 208 in a locked position against the cable 120. As can be seen from FIG. 3, the shoe 208 forces the cable 120 against a platen member 300 of the body 204. The toggle spring member 224 biases the shoe 208 against the cable 120 in the direction shown. The cable engaging surface of the shoe 208 is arcuate or curved in shape to resist frictionally displacement of the cable in the direction of the terminal device. Stated another way, the shoe is a self-energizing friction cleat that compresses the control cable against the stationary platen to prevent the cable's motion in a first direction 304 but not in a second (reverse or opposing) direction 308. The cable 120 has freedom of movement in the second direction 308. “Self-energizing” means the frictional force acting between the shoe and the cable attempts to move the shoe in a direction (the first direction 304), which further increases the friction force. Once the shoe and cable come into light contact, the interaction escalates resulting in the cable being solidly fixed and immovable against the fixed platen 300.

The self-energizing friction shoe or cleat 208 is preferably fabricated from a material that offers good abrasion resistance when used to grip or act against rough steel cables. In a preferred embodiment, the shoe 208 is fabricated from stainless steel and/or carbon steel.

The geometrical design requirement that makes the cleat or shoe self-energizing in the system is illustrated in FIG. 14. With reference to FIG. 14, the reaction angle a between the base pivot 1400 of the shoe 1408 (which has a different shoe configuration) and the contact point 1404 on the cable 120 is designed such that the tangent of the angle a is less than the coefficient of friction μ between the shoe material and the cable material. This equation is as follows:
Tangent α≦μ
The equation is derived by summing moments about the shoe pivot. The self-energizing friction cleat or shoe applies frictional load to the control cable to prevent its motion in one direction only.

FIG. 4 depicts the cable lock device 124 in the unlocked position. The shoe 208 has been rotated in the direction shown and engaged the movement limiter 212. In this position, the shoe is disengaged completely from the Bowden cable, providing the cable with unhindered freedom of movement in both the first and second directions 304 and 308.

FIG. 5 depicts the cable lock device 124 with the cover plate in position. The user moves the shoe between the engaged or locked (second) and disengaged or unlocked (first) positions by manipulating the actuation lever 222, which projects from the cover plate 228.

Apart from the shoe, the parts are sufficiently strong so as not to deform unacceptably under full mechanical loads. If possible, they should be designed with materials, such as aluminum alloys, that make them lightweight. The lightweight can be important as the unit is carried on the human body as a component in a prosthesis or orthotic brace.

The device assumes the cable has been installed through the inlet and outlet guides in the cable lock device, is relatively clean, and is not heavily lubricated with grease. If the cable is greased, the coefficient of friction will decrease, and the brake, while applying some force, might not achieve the full degree of self-energizing action desired.

In operation, the user, with the harness 104 engaged with his or her shoulders, uses scapular abduction to displace the cable 120 in the desired direction. When the cable 120 is in the desired position, the user uses the hand on his or her other arm to move the actuation lever 222 so that the shoe 208 is in the locked position. Alternatively, the user can move the lever to the locked position before the cable is at the desired position. The user can then use scapular abduction to move the cable in the second direction 308 until the cable is in the desired position. When the user has completed the desired task and seeks to release the grip, he or she moves the lever 222 so that the shoe 208 is in the unlocked position.

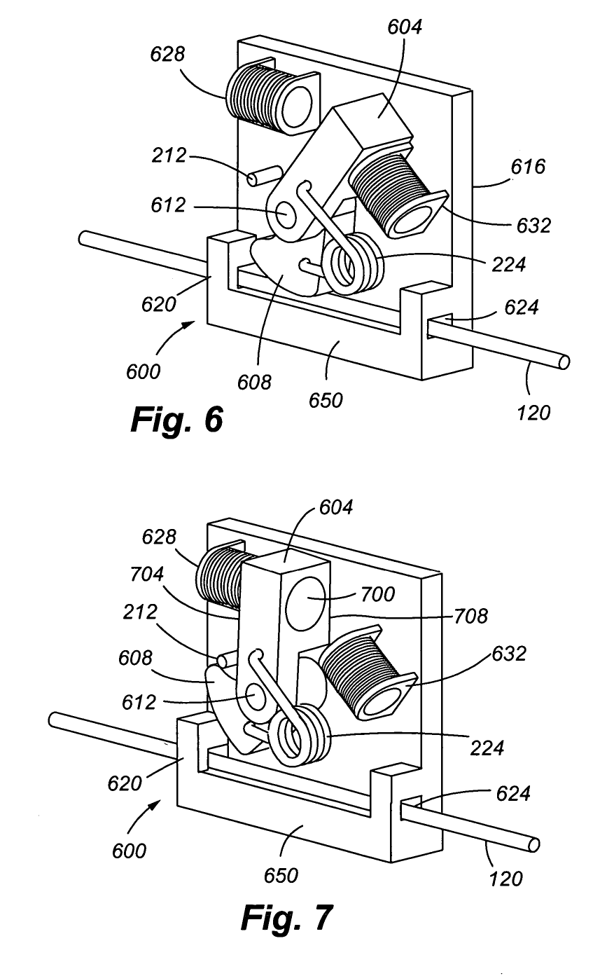

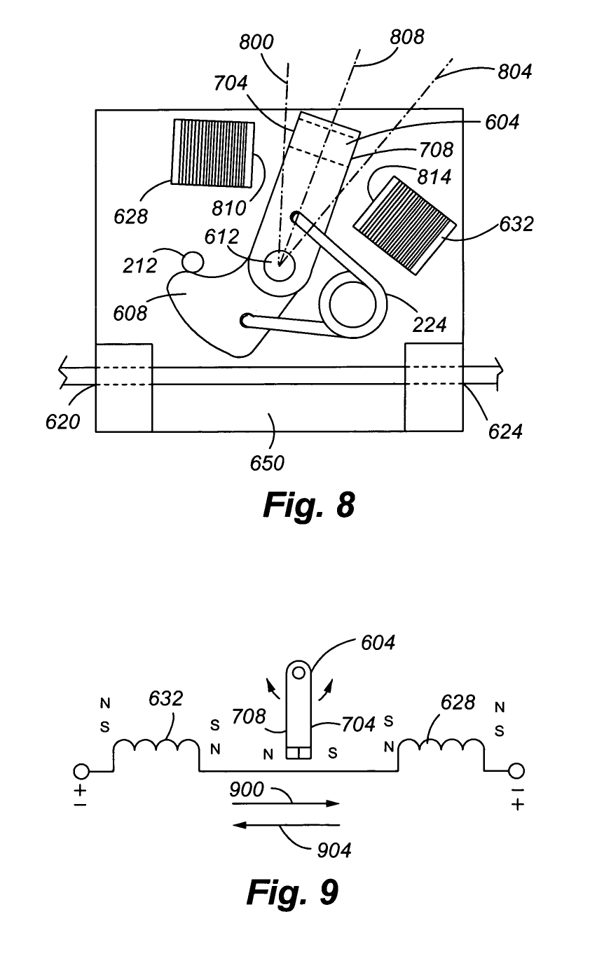

A hybrid or electromechanical cable lock device of the second embodiment will now be discussed with reference to FIGS. 6-9. As shown in FIGS. 6 and 7, the cable lock device 600 includes a toggle arm lever 604 and shoe 608 pivotably or rotatably mounted about the kingpin 612, the over-the-center spring member 224, the movement limiter 212, the body 616 having an inlet cable guide 620 and outlet guide 624 for the cable 120, and first and second biasing coils, or electromagnets, 628 and 632 for displacing the toggle arm lever 604 between first and second (bi-stable) positions 800 and 804 (FIG. 8). The toggle arm lever 604 includes a magnetic member 700 (FIG. 7) passing through the toggle arm lever 604, such that oppositely polarized faces of the magnet are exposed on each of the first and second sides 704 and 708 of the lever 604. In the absence of the spring member 224, the lever 604 and shoe 608 are rotate independently about the kingpin 612. The spring member 224, however, biases the shoe such that, when the lever 604 is in the first position 800, the shoe is unlocked and permits the cable 120 to move in both the first and second directions 304 and 308, and, when the lever 604 is in the second position 804, the shoe is locked and permits the cable 120 to move only in the second direction. In the second (lever) position, the shoe and stationary platen 650 prevent the cable from moving in the first direction. The spring member 224 thus acts as an over-the-center snap toggle mechanism and holds the toggle in either position until the electromagnet is energized and pushes the lever (which contains a permanent magnet) to the other position.

The first and second coils 628 and 632 define an electromagnetic toggle that moves the shoe into and out of contact with the cable. As noted, the toggle has first and second settings, with the first setting displacing the lever 604 to the first (unlocked) position 800 and the second setting displacing the lever 604 to the second (locked) position 804. As will be appreciated, electromagnetic coils, when energized, create a magnetic force, which moves the toggle from one coil to the other to change the device's state.

The operation of the electromagnetic toggle will now be described with reference to FIGS. 6-9. To cause the lever 604 to move to the first (unlocked) position 800, an electric current flows in the first direction 900 through the series connected first and second coils 628 and 632. The face 810 of the first coil 628 and the face 814 of the second coil 632 are polarized the same (e.g., both north or south). As can be seen in FIG. 9, the magnet in the lever is oriented such that the magnet facing surface on the face 708 is polarized as north and the magnet facing surface on the face 704 is polarized as south. The face 810 of the first coil 628 and the face 814 of the second coil 632 are polarized as north. Accordingly, the lever is caused to move towards the first coil 628, or to the first position 800. Likewise to cause the lever 604 to move to the second (locked) position 804, the polarity of the battery source is changed to cause an electrical current to flow in the second direction 904 through the series connected first and second coils 628 and 632. The face 810 of the first coil 628 and the face 814 of the second coil 632 are polarized as south. Accordingly, the lever is caused to move towards the second coil 632, or to the second position 804. As will be appreciated, the third position 808 of the lever 604 is its most unstable.

As will be appreciated, the orientation of the magnet in the lever can be reversed, such that the south pole of the magnet is adjacent the face 708 and the north pole is adjacent the face 704. In that configuration, the electrical current flows are reversed to place the lever in the first and second positions.

The user can manipulate the shoe between the locked and unlocked states by moving an electrical switch on a control unit (not shown) between first and second positions to switch the polarity of the power source as shown with reference to the terminals of FIG. 9. The power source may be located at any desirable location on the user, with the harness being preferred.

As will be also appreciated, though both coils are shown as being energized simultaneously to get more force, with one pushing and the other attracting the permanent magnet in the lever, the coils may be on separate circuits so that they are energized at different times. In this configuration, one coil provides an attractive or repulsive force at a first time and the second coil provides an attractive or repulsive force at a second different time.

The toggle provides a simple way to control whether or not the cable is locked. A desirable aspect of this configuration is the device only requires that electrical energy be expended to change the cable lock device's state from locked to unlocked or vice-versa. This conserves energy, and is useful to extend the service life of batteries if they are used. Once locked, the friction locking shoe is self-energizing and does not require additional electrical energy.

As will be appreciated, the two-state toggle may be replaced with any electromechanical equivalent that does not require continuous power to maintain its state, such as a two-state solenoid or an electric motor and gear mechanism. In addition, the electrical part can be removed and a second, separate control cable used to control the toggle position.

Various biasing springs can be added to control the initial contact pressure of the shoe upon the cable and to add resistance to the toggle to prevent inadvertent state changes if the mechanism is subjected to vibration or impact. Different materials can be used to control the friction coefficient of the shoe and to account for different cable material (steel wire, polymer ropes and cables, etc.). In addition, it is envisioned to add switching electronics that automatically control the voltage polarity applied to the magnetic coils so that flip-flopping can be easily achieved by pressing only a single one-contact momentary button switch. Optimization of the electromagnetic coils is possible to ensure they deliver a maximum force “kick” to move the toggle for a certain selection of battery.

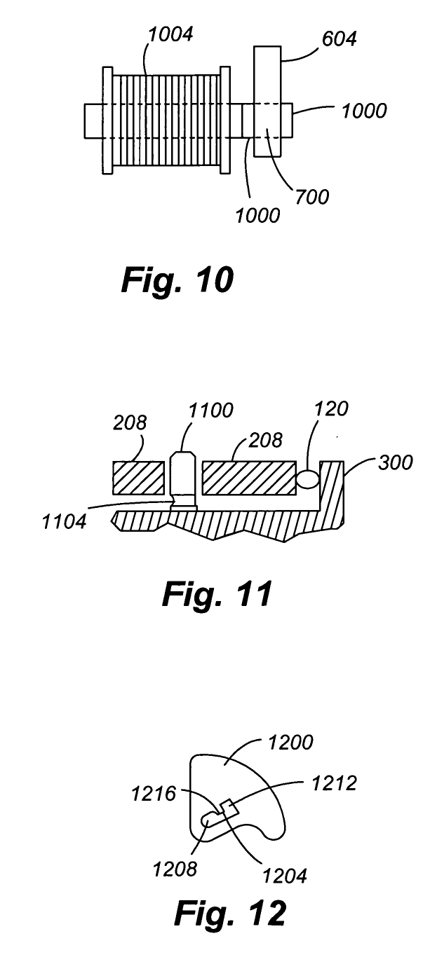

The permanent magnet 700 can be any desirable material. Preferably, the material is selected to provide a magnetic field able to supply a portative force on face contact of 1 lbf or less in normal use. In a particularly preferred configuration, the permanent magnet is a rare earth magnet, such as samarium cobalt or niobium magnets. A problem with such magnets, however, is that the attractive or portative holding force between the core of the coil, or electromagnet, and permanent magnet can exceed the repulsive force achievable using or arising in the energized electromagnets. To mitigate this problem, FIG. 10 shows that a diamagnetic, or non-magnetic, material 1000 is positioned between the face of the permanent magnet 700 and the adjacent electromagnet 1004 (such as coil 628 or 632). The non-magnetic material 1000 can be a shock absorbing, deformable, and/or elastic material, such as a foamed, polymeric, carbon bearing, conductive, or other type of material. As will be appreciated, the attractive force of a magnet is inversely proportional to the distance between the magnet and the attracting object. The material is preferably adhered to the opposing faces of the permanent magnet as shown in FIG. 10. The resulting offset distance between the electromagnetic core and permanent magnet reduces the permanent attractive force to a level below the maximum repulsive force of the electromagnet to ensure state changes are possible

Other embodiments will now be discussed with reference to FIGS. 11-13. These embodiments are designed to provide failure, and cable release, under a predetermined force to avoid injury to the operator in the event of cable lock device malfunction and/or an unintended or unanticipated catastrophic event.

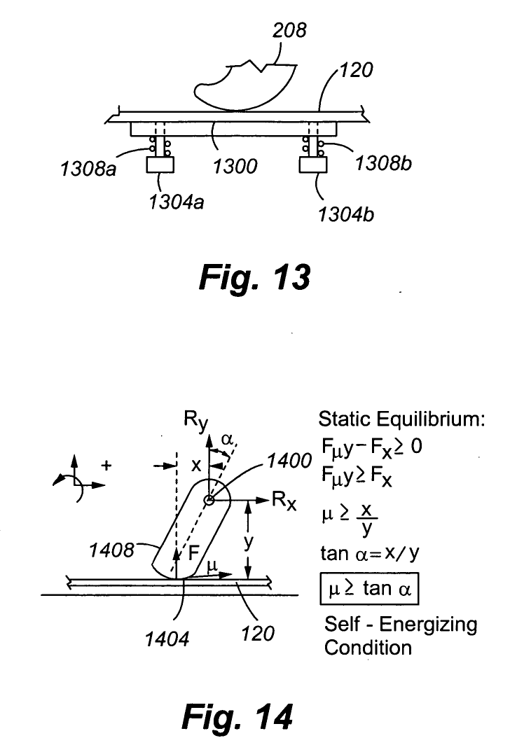

In one such embodiment shown in FIG. 13, the platen 1300 for backing the cable 120 against the shoe 208 is secured with screws 1304a,b and spring members 1308a,b, such as Belleville (spring) washers, to permit the platen's movement under extreme loads exerted by the cable on the shoe and kingpin to relieve the cable pinching force and enable cable release as an automatic safety feature.

In another embodiment shown in FIG. 11, the kingpin 1100 about which the shoe pivots includes a groove 1104, or stress riser, at its base. As a result, the kingpin 1100 is designed to fail through shear at a predetermined force exerted by the cable on the shoe and kingpin, or a corresponding internal stress. Alternatively, the kingpin 1100 is attached to a moveable mount (not shown) that is displaced under a predetermined force exerted by the cable on the shoe and kingpin to thereby relieve the cable pinching force.

In yet another embodiment shown in FIG. 12, the shoe 1200 includes an elongated slot 1204 that includes a first bore 1208 for the kingpin (not shown) and a second bore 1212 separated from the first bore 1208 by a projection 1216. Under the internal stress of the predetermined force on the shoe by the cable, the projection 1216 will fail causing the shoe to move on the kingpin to the second bore 1212. The resulting displacement of the shoe will cause release of the cable.

Finally, frictional dampers may be added to induce frictional drag on the toggle paddle or shoe as they rotate on the center post to introduce hysteresis or timing delays in the device's operation.

A number of variations and modifications of the invention can be used. It would be possible to provide for some features of the invention without providing others.

For example in one alternative embodiment, the lever 604 includes an electromagnet while the first and second coils are replaced by permanent magnets. The lever is displaced by passing current in one of two directions through the electromagnet causing an attractive and/ore repulsive force to displace the electromagnet.

In another alternative embodiment, the permanent magnet in the lever is replaced by an electromagnet that is connected, relative to the first and second electromagnets, to a separate circuit with the common power source.

In yet another alternative embodiment, a capstan-based approach permits free cable motion in one direction. A belt-band friction theory prevents backward movement. The drum could then use an electromagnetic clutch that is energized/deenergized to release the mechanism and permit free movement.

In yet another alternative embodiment, a motor drives a block of braking friction material into contact with the cable and effectively pinches it against an immovable or stationary platen. The principle is the same as clamping the cable in a vise.

In yet another embodiment, a lock- or coverplate is skewed by an actuator to lock the cable. This approach may or may not permit free motion in one direction. The idea is to pass the control cable through a closely matched hole in a plate. As long as the hole axis and the cable axis are aligned the cable will slip freely. If the plate is canted, the edges of the hole will be forced against the cable diameter, locking the cable so as to prevent relative motion.

In yet a further embodiment, a split-collet approach clamps down on the cable when it is pulled into a tapered, conical seat. A motor or other actuator then opens or changes the seat in a manner that relieves the collet's clamping action on the cable allowing it to freely slide through.

In yet another embodiment, a mechanism that operates like a ball point pen retraction system toggles between latched and unlatched states each time the cable is pulled through a full excursion cycle.

Each of the above approaches has various strengths and weaknesses relative to the others, but they could be made to work effectively with some engineering development.

In other applications, the principles of the present invention can be used in any mechanical system where a control cable must be prevented from moving in one direction when energized or “locked” while still allowing free cable motion in the opposing direction. When deenergized or “unlocked”, the cable may move freely in either direction unhindered.

The present invention, in various embodiments, includes components, methods, processes, systems and/or apparatus substantially as depicted and described herein, including various embodiments, subcombinations, and subsets thereof. Those of skill in the art wilI understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A method for operating a prosthetic and/or orthotic device, comprising:
(a) manipulating a cable lock device to be in a first mode, the first mode allowing a Bowden cable to move freely in first and second opposing directions; and
(b) manipulating the cable lock device to be in a second mode, the second mode inhibiting the Bowden cable from moving in the first direction but allowing the Bowden cable to move freely in the second direction.

2. The method of claim 1, wherein the cable lock device comprises a platen and a friction shoe positioned on either side of a section of the Bowden cable and an over-the-center spring member engaging the shoe and wherein the over-the-center spring member biases the shoe against the Bowden cable in the second mode.

3. The method of claim 2, wherein a surface of the shoe engaging the cable is arcuate in shape, wherein the shoe rotates about a kingpin, and wherein, in the first mode, the shoe is rotated out of contact with the cable.

4. The method of claim 3, wherein the following equation is true:
Tangent α≦μ
where α is an angle between lines intersecting the kingpin and a point of contact of the shoe with the cable and perpendicular to the platten.

5. The method of claim 2, wherein the cable lock device further comprises a lever including a magnetic member, the shoe and lever rotating with respect to one another, and at least one electromagnet to displace the lever between first and second positions, wherein, when the lever is in the first position, the device is in the first mode, and wherein, when the lever is in the second position, the device is in the second mode.

6. The method of claim 5, wherein the cable lock device comprises first and second spaced apart electromagnets, wherein the magnetic member in the lever is a permanent magnet, and wherein the shoe and lever rotate about a common axis of rotation.

7. The method of claim 6, wherein the over-the-center spring member engages both the lever and the shoe, wherein the lever is bi-stable, wherein the first and second electromagnets are electrically connected in series, wherein, when current flows through the electromagnets in one direction the lever is displaced towards the first electromagnet and, when the current flows through the electromagnets in an opposing direction, the lever is displaced towards the second electromagnet.

8. The method of claim 7, wherein at least one surface of lever contacts at least one of the first and second electromagnets, wherein the at least one surface is adjacent to the magnetic member, and wherein the at least one surface comprises a diamagnetic material to provide a space between the magnetic member and the at least one of the first and second electromagnets.

9. The method of claim 2, wherein the platen is spring loaded, whereby, when a force exerted by the cable on the platen exceeds a selected level, the platen is displaced, thereby permitting the cable lock device to enter automatically the first mode from the second mode.

10. The method of claim 3, wherein the shoe comprises first and second bores separated by a projection, wherein the kingpin is in the first bore and is separated from the second bore by the projection, and wherein, when a force exerted by the cable on the shoe exceeds a selected level, the projection fails and the kingpin moves into the second bore, thereby permitting the cable lock device to enter automatically the first mode from the second mode.

11. The method of claim 3, wherein the kingpin comprises a stress riser, whereby the kingpin fails when the force exerted by the cable on the shoe exceeds a selected level, thereby permitting the cable lock device to enter automatically the first mode from the second mode.

12. A cable lock device, comprising:
a body; and
a frictional shoe operable to engage a Bowden cable, wherein, in a first mode, the Bowden cable moves freely in first and second opposing directions and, in a second mode, the shoe inhibits the Bowden cable from moving in the first direction while allowing the Bowden cable to move freely in the second direction.

13. The device of claim 12, wherein the body comprises a platen, the platen and shoe being positioned on either side of a section of the Bowden cable, and further comprising an over-the-center spring member engaging the shoe, wherein the over-the-center spring member biases the shoe against the Bowden cable in the second mode.

14. The device of claim 13, wherein a surface of the shoe engaging the cable is arcuate in shape, wherein the shoe rotates about a kingpin, and wherein, in the first mode, the shoe is rotated out of contact with the cable.

15. The device of claim 14, wherein the following equation is true:
Tangent α≦μ
where α is an angle between lines intersecting the kingpin and a point of contact of the shoe with the cable and perpendicular to the platten.

16. The device of claim 13, further comprising a lever including a magnetic member, the shoe and lever rotating with respect to one another, and at least one electromagnet to displace the lever between first and second positions, wherein, when the lever is in the first position, the device is in the first mode, and wherein, when the lever is in the second position, the device is in the second mode.

17. The device of claim 16, wherein the cable lock device comprises first and second spaced apart electromagnets, wherein the magnetic member in the lever is a permanent magnet, and wherein the shoe and lever rotate about a common axis of rotation.

18. The device of claim 17, wherein the over-the-center spring member engages both the lever and the shoe, wherein the lever is bi-stable, wherein the first and second electromagnets are electrically connected in series, wherein, when current flows through the electromagnets in one direction the lever is displaced towards the first electromagnet and, when the current flows through the electromagnets in an opposing direction, the lever is displaced towards the second electromagnet.

19. The device of claim 18, wherein at least one surface of lever contacts at least one of the first and second electromagnets, wherein the at least one surface is adjacent to the magnetic member, and wherein the at least one surface comprises a diamagnetic material to provide a space between the magnetic member and the at least one of the first and second electromagnets.

20. The device of claim 13, wherein the platen is spring loaded, whereby, when a force exerted by the cable on the platen exceeds a selected level, the platen is displaced, thereby permitting the cable lock device to enter automatically the first mode from the second mode.
